(19) United States
(12) Patent Application Publication
(10) Pub. No.: US 2009/0209819 A1
(43) Pub. Date: Aug. 20, 2009

(54) BENDING SECTION STRUCTURE OF ENDOSCOPE

(75) Inventors: Hideya Kitagawa, Hachioji-shi (JP); Yoshiaki Ito, Fuchu-shi (JP)

Correspondence Address: Frishauf, Holtz Goodman & Chick, P.C., 220 Fifth Avenue, 16th Floor, New York, NY, 10001-7708, US

(73) Assignee: Olympus Corporation, Tokyo (JP)

(21) Appl. No.: 12/430,299

(22) Filed: Apr. 27, 2009

Related U.S. Application Data
(63) Continuation of application No. PCT/JP2007/071055, filed on Oct. 29, 2007.

(30) Foreign Application Priority Data
Oct. 30, 2006 (JP) 2006-294917

(51) Int. Cl. A61B 1/008

(52) U.S. Cl. 600/142

(57) ABSTRACT

First stepped portions are provided on both sides of each of two front-side projection portions of a front end portion of a node ring. Second stepped portions are provided on both sides of each of two rear-side projection portions of a rear end portion of the node ring. At a time of bending of a bending section, when two front and rear node rings of a bending tube are rotated to a maximum rotational position, the pivotal angle α between the front and rear node rings is restricted to a predetermined restriction angle that prevents, when the bending section is bend-operated, an outer sheath tube from being bitten between the node rings, by the restriction due to contact between the second stepped portion of the front-side node ring and the first stepped portion of the rear-side node ring.

### CROSS REFERENCE TO RELATED APPLICATIONS

This is a Continuation Application of PCT Application No. PCT/JP2007/071055, filed Oct. 29, 2007, which was published under PCT Article 21(2) in Japanese.

This application is based upon and claims the benefit of priority from prior Japanese Patent Application No. 2006-294917, filed Oct. 30, 2006, the entire contents of which are incorporated herein by reference.

### BACKGROUND OF THE INVENTION

1. Field of the Invention

The present invention relates to a bending section structure of an endoscope in which a bending section that is bendable is provided on a distal end side of an insertion section which is inserted into the body.

2. Description of the Related Art

In general, in a flexible endoscope, a proximal-side operation section is provided on a proximal end side of an insertion section which is inserted into the body. The insertion section includes an elongated flexible tube section, a bending section which is bendable, and a distal-end rigid section. The bending section is provided continuous with a distal end portion of the flexible tube section. The distal-end rigid section is provided at a distal end of the insertion section. A proximal end portion of the flexible tube section is coupled to the operation section.

The bending section includes a plurality of node rings. The plurality of node rings are juxtaposed along the insertion direction of the insertion section. Front-side and rear-side node rings are coupled by support shaft portions, such as rivets, so that the front-side and rear-side node rings are rotatable. Further, distal end portions of four bending operation wires are fixed to a distal end portion of the bending section. The four bending operation wires bend-operate the bending section, for example, in four directions, namely, upward, downward, leftward and rightward directions. Proximal end portions of these bending operation wires extend to the proximal-side operation section through the inside of the flexible tube section.

The proximal-side operation section is provided with a bending operation mechanism section which bend-operates the bending section, for example, in four directions, namely, upward, downward, leftward and rightward directions. The proximal end portions of the four bending operation wires are coupled to the bending operation mechanism section. In addition, an up-and-down bending operation knob and a left-and-right bending operation knob are provided in the bending operation mechanism section. One of the four bending operation wires is pulled and operated in accordance with the rotating operation of the up-and-down bending operation knob or the left-and-right bending operation knob. The bending section is bend-operated in one of the four directions, i.e. the upward, downward, leftward and rightward directions, via the pulled bending operation wire, or is bend-operated in an arbitrary direction at an arbitrary angle by plural ones of the bending operation wires.

Jpn. Pat. Appln. KOKOKU Publication No. H8-17766 (patent document 1) shows an example of the bending section structure of a conventional endoscope. In this example, a pair of forward projection portions, which protrude forward, are projectingly provided on a front-end outer peripheral surface of each node ring at positions of 180°. In addition, a pair of backward projection portions, which protrude backward, are projectingly provided on a rear-end outer peripheral surface of each node ring at positions of 180°. These forward projection portions and backward projection portions are disposed at positions with a displacement of 90° in the circumferential direction. The backward projection portion of the front-side node ring and the forward projection portion of the rear-side node ring are overlapped (“overlap”), thereby forming overlap portions. A through-hole is formed in each overlap portion, and a rivet is inserted in the through-hole. In this state, by caulking the rivet, a rotational support shaft (support shaft portion), which rotatably couples the node rings, is formed. Thereby, a link structure, which bend-operates the bending section, for example, in the four directions, i.e. the upward, downward, leftward and rightward directions, is formed by the plural node rings.

Further, an outer sheath, such as a rubber tube, is coated on the outer peripheral surface of the bending tube in which the plural node rings are juxtaposed. In the bending tube of the endoscope of patent document 1, a protection net is interposed between the node ring and the outer sheath. In this structure, the protection net prevents the outer sheath from being bitten between the node rings of the bending tube, when one of the four bending operation wires is pulled and operated to bend-operate the bending section in one of the four directions, i.e. the upward, downward, leftward and rightward directions, or plural ones of the bending operation wires are pulled and operated to bend-operate the bending section in an arbitrary direction, in accordance with the rotating operation of the up-and-down bending operation knob or the left-and-right bending operation knob.

### BRIEF SUMMARY OF THE INVENTION

According to an aspect of the present invention, there is provided a bending section structure of an endoscope, comprising a bending section including a bending tube in which a plurality of node rings are juxtaposed along an insertion direction of an endoscope insertion section and are coupled such that front and rear the node rings are rotatable about support shaft portions, and an outer sheath which is formed of an elastic material in a circular cylindrical shape and is directly fitted over an outer periphery of the bending tube, wherein the bending section structure of the endoscope includes a bending wire for pulling and operating the Lending section, and the node rings of the bending tube are rotated and operated about the support shaft portions by the pulling operation of the bending wire, thereby bending the bending section, the node ring has bite prevention means for restricting, to a predetermined restriction angle, a pivotal angle between the front and rear node rings which rotate about the support shaft portions when the bend section is bend-operated, and the restriction angle is so set as not to exceed a bite limit thickness at which the outer sheath is not damaged when the outer sheath is bitten between the node rings.

In the above-described structure, at the time of the bending operation of the bending section, when the front and rear node rings are rotated about the support shaft portions, the bite prevention means of the node rings restricts the pivotal angle between the front and rear node rings to a predetermined restriction angle. Thus, the predetermined restriction angle corresponds to the maximum rotational position of the pivotal angle between the front and rear node rings. The restriction angle of the pivotal angle between the front and rear node rings restricts the pivotal angle by considering in advance the thickness at which bite of the outer sheath occurs in the state in which the outer sheath is buckled in the inside of bending and fitted between the node rings when the bending section is bend-operated, or the limit thickness at which the outer sheath is not damaged even if the outer sheath is bitten. Hence, the minimum node ring interval between the node rings, which are combined when the bending tube is coupled, is restricted so as not to reach the limit thickness. Thereby, when the bending section is bend-operated, the front and rear node rings rotate to the maximum rotational position about the support shaft portions at the part that is located on the inside of the bending of the bending section, and the outer sheath is prevented from being bitten between the node rings and damaged, even in the state in which the distance between the node rings is narrowed.

It is preferable that the node ring includes an annular node ring body, a front-side hinge base which is forwardly protectingly provided at a front end portion of the node ring body, and a rear-side hinge base which is rearwardly projectingly provided at a rear end portion of the node ring body, the support shaft portion includes a pivotal support shaft which is pivotally coupled to an overlap part between the rear-side hinge base of the node ring, which is disposed on a front side, and the front-side hinge base of the node ring, which is disposed on a rear side, the bite prevention means includes an angle restriction member which is provided on the node ring, and the angle restriction member restricts, to the restriction angle, the pivotal angle between the front and rear node rings which rotate about the pivotal support shafts when the bend section is bend-operated.

In the above-described structure, at the time of the bending operation of the bending section, when the front and rear node rings are rotated about the support shaft portions, the angle restriction member of the node ring, which is the bite prevention means, restricts the pivotal angle between the front and rear node rings to a predetermined restriction angle.

It is preferable that the angle restriction member restricts the restriction angle such that a minimum node ring interval between the front and rear node rings becomes greater than a bite thickness of the outer sheath that is fitted between the node rings when the bending section is bend-operated.

In the above-described structure, at the time of the bending operation of the bending section, when the front and rear node rings are rotated about the support shaft portions, the angle restriction member restricts the pivotal angle between the front and rear node rings to a predetermined restriction angle. Thereby, the minimum node ring interval between the front and rear node rings is set to be greater than the bite thickness of the outer sheath, at which the outer sheath is fitted between the node rings and is damaged.

It is preferable that the angle restriction member includes, at least on one of the rear-side hinge base and the front-side hinge base, an abutment portion at which the rear-side hinge base and the front-side hinge base are abutted at the restriction angle when the bending section is bend-operated.

In the above-described structure, at the time of the bending operation of the bending section, when the front and rear node rings are rotated about the support shaft portions, the abutment portion of the rear-side hinge base and the abutment portion of the front-side hinge base are put in contact. Thereby, the pivotal angle between the front and rear node rings is restricted to a predetermined restriction angle.

It is preferable that the abutment portion includes, at each of the rear-side hinge base and the front-side hinge base, a stepped portion for abutment at the restriction angle at a time when the bending section is bend-operated.

In the above-described structure, at the time of the bending operation of the bending section, when the front and rear node rings are rotated about the support shaft portions, the stepped portion of the rear-side hinge base and the stepped portion of the front-side hinge base are put in contact. Thereby, the pivotal angle between the front and rear node rings is restricted to a predetermined restriction angle.

It is preferable that the angle restriction member includes a coupling band which is passed between the front and rear node rings, and the coupling band is stretched on an opening side of the node rings when the bending section is bend-operated, thereby restricting a maximum node ring interval between the front and rear node rings at the restriction angle.

In the above-described structure, at the time of the bending operation of the bending section, when the front and rear node rings are rotated about the support shaft portions, the coupling band between the front and rear node rings is stretched on the opening side of the node rings, thereby restricting the maximum node ring interval between the front and rear node rings at the restriction angle. Thus, the pivotal angle between the front and rear node rings is restricted to a predetermined restriction angle.

It is preferable that first protrusion portions for positioning are forwardly projectingly provided on both sides of the front-side hinge base of the node ring, and second protrusion portions for positioning are rearwardly projectingly provided on both sides of the rear-side hinge base of the node ring, and the angle restriction member restricts the restriction angle by putting the second protrusion portion of the rear-side hinge base and the first protrusion portion of the front-side hinge base at the restriction angle when the bending section is bend-operated.

In the above-described structure, at the time of the bending operation of the bending section, when the front and rear node rings are rotated about the support shaft portions, the second protrusion portion of the rear-side hinge base and the first protrusion portion of the front-side hinge base are put in contact at the restriction angle. Thereby, the pivotal angle between the front and rear node rings is restricted to a predetermined restriction angle.

### BRIEF DESCRIPTION OF THE SEVERAL VIEWS OF THE DRAWING

FIG. 1 schematically shows the entire structure of a general endoscope to which a bending section structure of an endoscope according to a first embodiment of the present invention is applied;

FIG. 2 schematically shows an internal structure of a distal-end rigid section of the endoscope according to the first embodiment;

FIG. 3 is a cross-sectional view, taken along line III-III in FIG. 1, showing a cross section of a bending section of the endoscope according to the first embodiment;

FIG. 4 is a side view showing a state in which node rings of the bending section of the endoscope according to the first embodiment are juxtaposed;

FIG. 5 is a perspective view showing one node ring of the bending section of the endoscope according to the first embodiment;

FIG. 6 is a transverse cross-sectional view of parts of wire guides of the node ring of the bending section of the endoscope according to the first embodiment;

FIG. 7 is a perspective view of a main part, showing a state in which two node rings of the bending tube are rotated to a maximum rotation position when the bending section of the endoscope according to the first embodiment is bent;

FIG. 8 is a side view of a main part, showing a state in which two node rings of the bending tube are rotated to a maximum rotation position when the bending section of the endoscope according to the first embodiment is bent;

FIG. 9 is a longitudinal partial cross-sectional view of a main part, showing a state in which the bending section of the endoscope according to the first embodiment is held in a non-bent state;

FIG. 10 is a longitudinal partial cross-sectional view of a main part, illustrating a deformed state of an outer sheath tube when the bending section of the endoscope according to the first embodiment is bent;

FIG. 11 is a longitudinal partial cross-sectional view of a main part, showing a state in which a bending section of an endoscope according to a second embodiment of the present invention is held in a non-bent state;

FIG. 12 is a longitudinal partial cross-sectional view of a main part, illustrating a deformed state of an outer sheath tube when the bending section of the endoscope according to the second embodiment is bent;

FIG. 13 is a side view of a main part, showing a state in which two node rings of a bending tube are rotated to a maximum rotation position when a bending section of an endoscope according to a third embodiment of the present invention is bent;

FIG. 14A is a longitudinal partial cross-sectional view of a main part, showing a state in which the bending section of the endoscope according to the third embodiment is held in a non-bent state; and

FIG. 14B is a longitudinal partial cross-sectional view of a main part, illustrating a deformed state of an outer sheath tube when the bending section of the endoscope according to the third embodiment is bent.

### DETAILED DESCRIPTION OF THE INVENTION

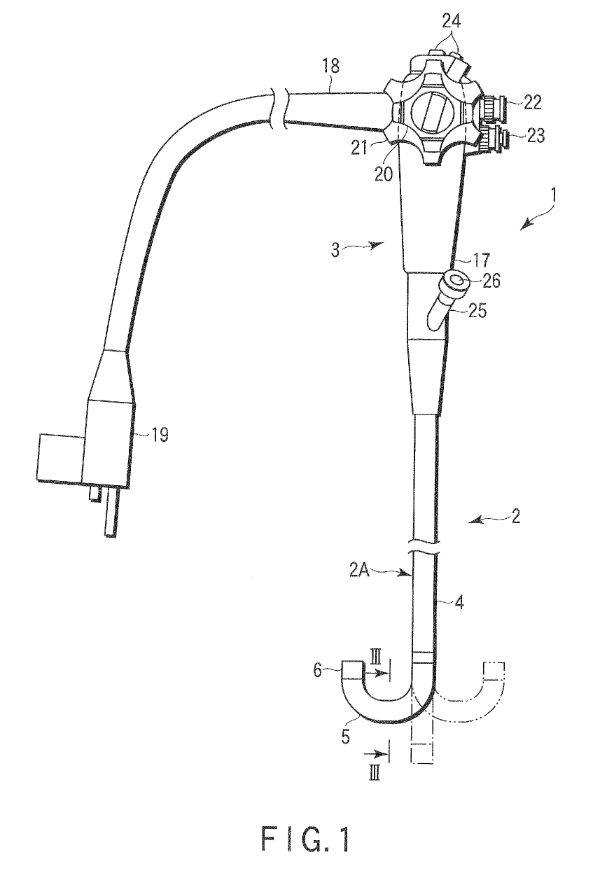

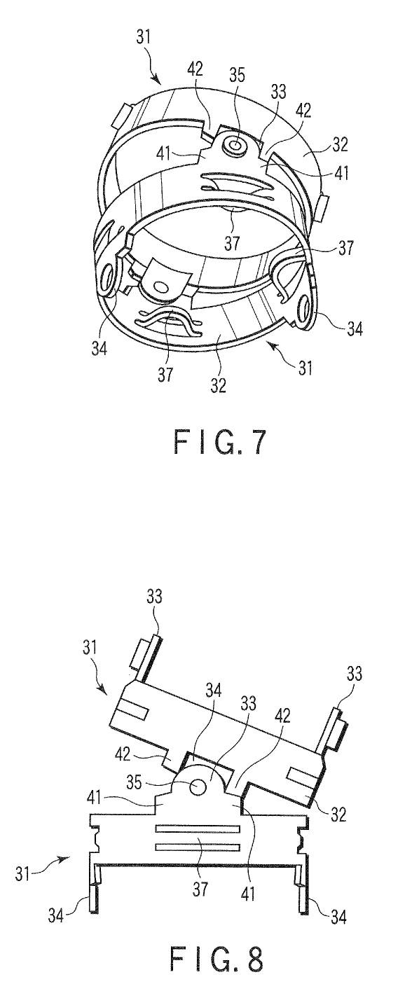

A first embodiment of the present invention will now be described with reference to FIG. 1 to FIG. 10. FIG. 1 shows an example of a flexible endoscope 1, such as a colonoscope, to which a bending section structure of an endoscope according to the present embodiment is applied. The endoscope 1 includes an elongated insertion section 2 which is inserted into the body, and an operation section 3 which is coupled to a proximal end portion of the insertion section 2.

A main body 2A of the insertion section 2 includes an elongated flexible tube section 4, a bending section 5, and a distal-end rigid section 6. A proximal end portion of the bending section 5 is coupled to a distal end portion of the flexible tube section 4. A proximal end portion of the distal-end rigid section 6 is coupled to a distal end portion of the bending section 5. The bending section 5 can be bend-operated from a normal straight state as indicated by a dot-and-dash line in FIG. 1, to a bent state as indicated by a solid line or a two-dot-and-dash line in FIG. 1.

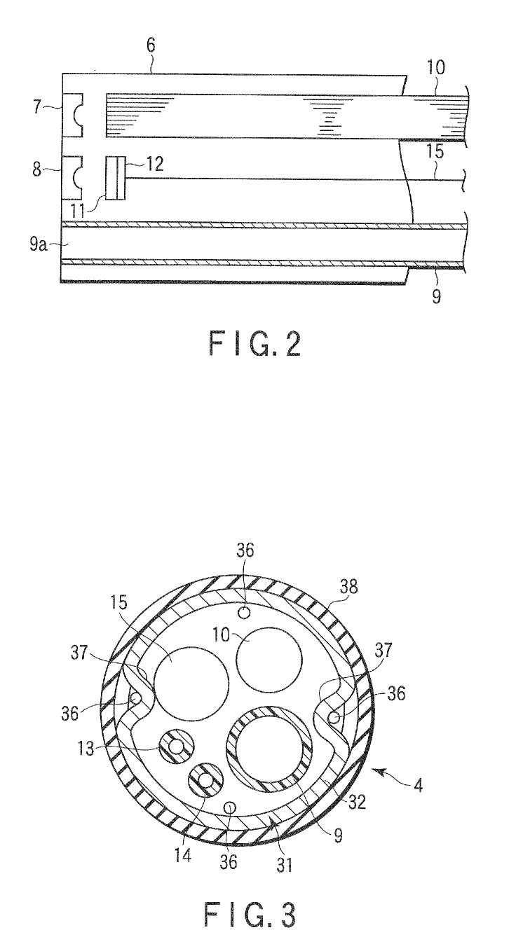

As shown in FIG. 2, a distal-end face of the distal-end rigid section 6 is provided with an illumination lens 7 of an illumination optical system, an objective lens 8 of an observation optical system, a distal-end opening portion 9a of a therapeutic device insertion channel 9, and an air/water feed nozzle (not shown).

In the distal-end rigid section 6, a distal end portion of a light guide fiber 10 is fixed behind the illumination lens 7. Further, an image pickup element 11, such as a CCD, and a connection circuit board 12 thereof are fixed behind the objective lens 8. A distal end portion of an image guide fiber (not shown), in place of the image pickup element 11, may be fixed, and the endoscope 1 may be configured as a fiber scope, and not as an electronic scope. Further, in the distal-end rigid section 6, a distal end portion of the therapeutic device insertion channel 9 and distal end portions of an air-feed tube 13 (see FIG. 3) and a water-feed tube 14 (see FIG. 3), which are connected to the air/water feed nozzle, are fixed.

As shown in FIG. 3, built-in parts of the insertion section 2, which have their distal end portions fixed to the above-described distal-end rigid section 6, namely, the light guide fiber 10, a cable 15, such as a signal cable of the image pickup element 11, an image guide fiber (not shown) in the case of a fiber scope, the therapeutic device insertion channel 9, the air-feed tube 13 and the water-feed tube 14 pass through the flexible tube section 4 from the bending section 5, and extend to the proximal end side of the flexible tube section 4.

The operation section 3 is coupled to a proximal end portion of the flexible tube section 4. The operation section 3 is provided with a hold section 17 which is held by a surgeon. A proximal end portion of a universal cord 18 is coupled to the hold section 17. A distal end portion of the universal cord 18 is connected to a connector section 19. The connector section 19 is connected to, e.g. a light source device and a video processor (not shown).

Further, the operation section 3 is provided with an up-and-down bending operation knob 20 and a left-and-right bending operation knob 21 for bend-operating the bending section 5, a suction button 22, an air/water feed button 23, various switches 24 for endoscopy, and a therapeutic device insertion section 25. The therapeutic device insertion section 25 is provided with a therapeutic device insertion hole 26. The therapeutic device insertion hole 26 is connected to a proximal end portion of the therapeutic device insertion channel 9 that is provided in the insertion section 2. An endoscopic therapeutic device (not shown) is inserted from the therapeutic device insertion hole 26 of the endoscope 1 into the therapeutic device insertion channel 9, and is pushed to the distal-end rigid section 6 side. Then, the endoscopic therapeutic device is projected to the outside from the distal-end opening portion 9a of the therapeutic device insertion channel 9.

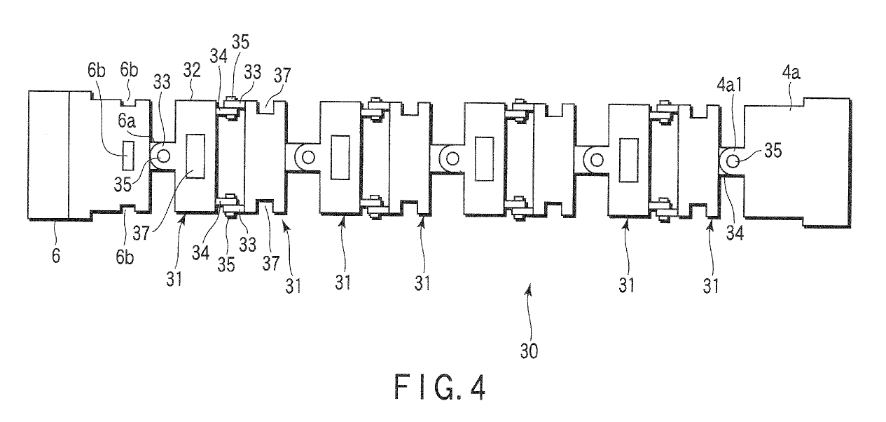

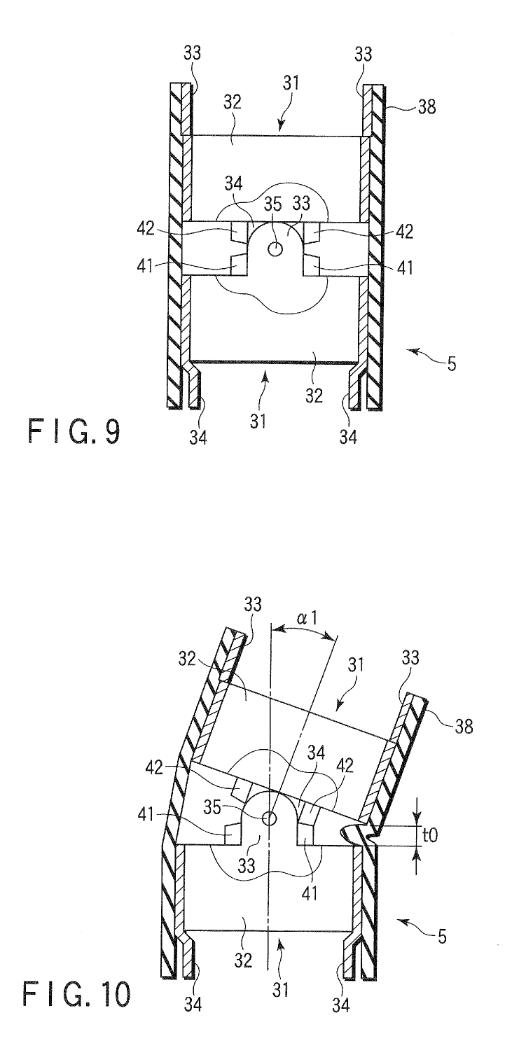

The bending section 5 according to the present embodiment, as shown in FIG. 4 and FIG. 9, includes a bending tube 30 and an outer sheath tube 38 (to be described later). The bending tube 30 includes a plurality of node rings 31. As shown in FIG. 4, the plural node rings 31 are juxtaposed along the insertion direction (axial direction) of the insertion section 2 of the endoscope 1. Front-side and rear-side node rings 31 are coupled to be rotatable about rivets (support shaft portions) 35, which are to be described later. The outer sheath tube 38 is formed of an elastic material in a cylindrical shape, and is directly fitted over the outer periphery of the bending tube 30. The outer sheath tube 38 is formed by injection molding in a cylindrical shape of an elastic material such as a thermoplastic elastomer (styrene, olefin or urethane). Thereby, the entire outer surface of the bending section 5 is covered with the outer sheath tube 38. The formation of the thermoplastic elastomer is not limited to injection molding, and various formation methods, such as casting, extrusion, and blowing, may be applied. Besides, the elastic material is not limited to the thermoplastic elastomer, and may be a rubber material.

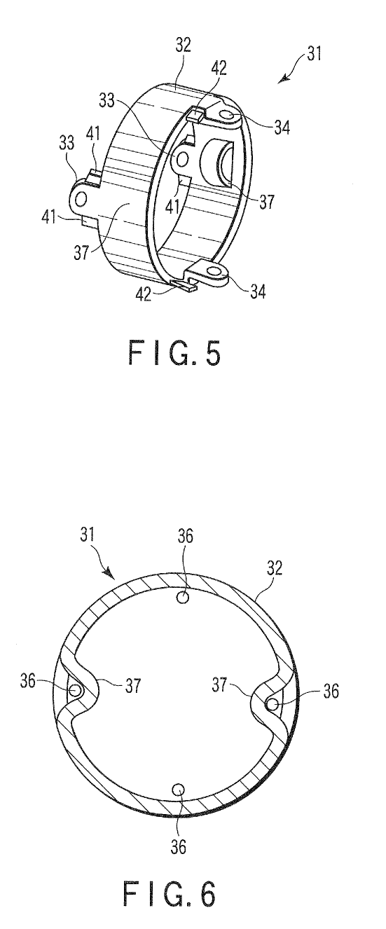

As shown in FIG. 5, each node ring 31 has a circular cylindrical node ring main body 32. The node ring main body 32 is formed of, e.g. a metal thin plate press body or a cast body. Two projection portions (front-side hinge bases) 33 are disposed at a distal end portion of the node ring main body 32 at positions spaced apart by 180° in the circumferential direction. The two projection portions 33 are formed by forwardly projecting parts of the outer peripheral surface of the node ring main body 32. Two projection portions (rear-side hinge bases) 34 are disposed at a rear end portion of the node ring main body 32 at positions spaced apart by 180° in the circumferential direction. The two front-side projection portions 33 and two rear-side projection portions 34 are disposed at positions with a displacement of 90° in the circumferential direction. The two rear-side projection portions 34 are formed by backwardly projecting parts of the outer peripheral surface of the node ring main body 32 with a step substantially corresponding to the thickness of the projection portion 33.

In each node ring 31, first stepped portions 41 (to be described later) are formed on both sides of each of the two front-side projection portions 33. Similarly, second stepped portions 42 (to be described later) are formed on both sides of each of the two rear-side projection portions 34. The details of the first stepped portions 41 and second stepped portions 42 will be described with reference to FIG. 9. In FIG. 4, depiction of each stepped portion 41, 42 is omitted.

The plural node rings 31, which are juxtaposed in the bending section 5, are so coupled as to be rotatable in the following manner. The two rear-side projection portions 34 of the front-side node ring 31 and the two front-side projection portions 33 of the rear-side node ring 31 are coupled via rivets 35 which are inserted in holes formed in the respective projection portions 33 and 34. Thereby, the front-side node ring 31 and rear-side node ring 31 are pivotally supported in a manner to be rotatable about the rivets 35, and journal portions are formed therebetween, with the rivets 35 being used as rotational support shafts.

Further, the two front-side projection portions 33 of the foremost node ring 31 of the bending section 5 are similarly coupled to two projection portions 6a which are rearwardly projectingly provided on a rear end portion of the distal-end rigid section 6 via rivets 35. Thereby, the foremost node ring 31 of the bending section 5 is pivotally supported on the rear end portion of the distal-end rigid section 6 in a manner to be rotatable about the rivets 35. In addition, the two rear-side projection portions 34 of the rearmost node ring 31 of the bending section 5 are similarly coupled via rivets 35 to two projection portions 4a1 which are forwardly projectingly provided on a circular cylindrical coupling member 4a which is disposed at the distal end of the flexible tube section 4. Thereby, the rearmost node ring 31 of the bending section 5 is pivotally supported on the coupling member 4a at the distal end of the flexible tube section 4 in a manner to be rotatable about the rivets 35.

In the bending section 5 of the present embodiment, the directions of the rivets 35, which function as rotational support shafts that couple the plural node rings 31, are alternately arranged with a displacement of 90° between front-side and rear-side node rings 31. Thereby, the entire bending section 5 can be bent in four directions, i.e. the upward, downward, leftward and rightward directions.

As shown in FIG. 3 and FIG. 6, in the bending section 5, four operation wires (bending wires) 36 for bend-operating the entire bending section 5 in the four directions, i.e. the upward, downward, leftward and rightward directions, are provided. Distal end portions of the four operation wires 36 are fixed to the rear end portion of the distal-end rigid section 6. The operation wires 36 are fixed by silver soldering to recess portions 6b which are formed by cutting and inwardly bending those portions of the distal-end-side peripheral wall part, which correspond to the projection portions 6a, by a pressing process. The recess portions 6b are formed at four locations with a displacement of 90° in the circumferential direction. The distal end portions of the operation wires 36 may be fixed to recess portions (not shown) which are formed in the foremost node ring 31.

As shown in FIG. 6, two wire guides (wire receivers) 37, which project inward, are provided on the peripheral wall part of the node ring main body 32 of each node ring 31. The wire guides 37 are formed by cutting and bending, and projecting and raising, portions of the peripheral wall part of the node ring main body 32 from the outer peripheral side to the inner peripheral side by a pressing process. Either up-and-down operation wires 36 or left-and-right operation wires 36 are passed through the wire guides 37.

Proximal end portions of the up-and-down operation wires 36 and left-and-right operation wires 36 pass through the flexible tube section 4 from the bending section 5, and extend into the operation section 3. In the operation section 3, there are provided an up-and-down bending operation mechanism (not shown) which is driven by the up-and-down bending operation knob 20, and a left-and-right bending operation mechanism (not shown) which is driven by the left-and-right bending operation knob 21. The proximal end portions of the up-and-down operation wires 36 are coupled to the up-and-down bending operation mechanism. Similarly, the proximal end portions of the left-and-right operation wires 36 are coupled to the left-and-right bending operation mechanism. In accordance with the rotational operations of the up-and-down bending operation knob 20 and the left-and-right bending operation knob 21, the respective operation wires 36 are pulled and driven. Thereby, the bending section 5 is remotely bend-operated from the normal straight state (non-bent state) in which the bend angle is 0°, to the bend-operated shape at an arbitrary bend angle in the upward, downward, leftward or rightward direction.

In the present embodiment, as shown in FIG. 9 that has been described above, the first stepped portions (bite-prevention means) 41 for angle restriction are formed on both sides of each of the two front-side projection portions 33. The first stepped portion 41 restricts a pivot angle α between the two front-side and rear-side node rings 31 of the bending tube 30 when the bending section 5 is bent. Further, the second stepped portions (bite-prevention means) 42 for angle restriction are formed on both sides of each of the two rear-side projection portions 34. The second stepped portion 42 restricts the pivot angle α.

FIG. 9 shows the state in which the bending section 5 is kept in the non-bent state. In the state shown in FIG. 9, the two rear-side second stepped portions 42 of the front-side node ring 31 which are positioned on the front end side of the bending section 5, and the two first stepped portions 41 of the rear-side node ring 31, which are positioned on the rear end side of the bending section 5, are separated in the non-contact state. FIG. 10 shows the state in which the two front-side and rear-side node rings 31 of the bending tube 30 are rotated to the maximum degree when the bending section 5 is bent. In the state shown in FIG. 10 one of the two rear-side second stepped portions 42 of the front-side node ring 31 and one of the two first stepped portions 41 of the rear-side node ring 31 are abutted and put in contact. Thereby, the pivot angle α between the front and rear node rings 31 in the case where the two front and rear node rings 31 are rotated to the maximum when the bending section 5 is bent is restricted to a predetermined restriction angle α1.

The predetermined restriction angle α1 of the pivot angle α is set at such an angle as to be able to prevent the outer sheath tube 38 from being bitten between the node rings 31, which rotate about the rivets 35 when the bending section 5 is bent, and being damaged. For example, in the bending tube 30 of the present embodiment, in the case (non-restriction time) where there is no restriction due to contact between the second stepped portion 42 of the front-side node ring 31 and the first stepped portion 41 of the rear-side node ring 31 at the time of bending of the bending section 5, the maximum pivotal angle α0 between the front and rear node rings 31 is set at 26.3°. The position in the maximum bend state of the bending section 5 at the time of bending of the bending section 5 is the position in which the end edge portions of the front and rear node rings 31 on the inner side of the bending of the bending section 5 are put in contact. The pivotal angle α between the front and rear node rings 31 at this time is the maximum pivotal angle α0. Further, the outer sheath bite limit thickness t0, at which the outer sheath tube 38 is not damaged in the state in which the bending portion of the outer sheath tube 38 is clamped between the end edge portions of the front and rear node rings 31 on the inner side of the bending of the bending section 5 at the time of bending of the bending section 5, as shown in FIG. 10, is set at 0.35 mm. Under this condition, the pivotal angle (restriction angle) α between the front and rear node rings 31 of the bending tube 30 at the time of bending of the bending section 5 is set at 23.0° that is less than α0 (α<α0) by the restriction due to the contact between the second stepped portion 42 of the front-side node ring 31 and the first stepped portion 41 of the rear-side node ring 31 at the time of bending of the bending section 5.

Next, the operation of the above-described structure is described. When the endoscope 1 of the present embodiment is used, the respective operation wires 36 are pulled and driven in accordance with the rotational operation of the up-and-down bending operation knob 20 and the left-and-right bending operation knob 21 of the operation section 3. Thereby, the bending section 5 is remotely bend-operated from the normal straight state (non-bent state) in which the bend angle is 0°, as shown in FIG. 9, to the bend-operated shape at an arbitrary bend angle in the upward, downward, leftward or rightward direction, as shown in FIG. 10.

When the bending section 5 is bend-operated, as shown in FIG. 10, at the part that is positioned on the outside of the bending of the bending section 5, the distance at the gap portion between the node rings 31 increases in accordance with the rotational operation of each node ring 31. Thereby, tensile force acts on the outer sheath tube 38, and the outer sheath tube 38 is stretched in an elastically deformed state. At the same time, at the part that is positioned on the inside of the bending of the bending section 5, the distance at the gap portion between the node rings 31 decreases. Thereby, compressive force acts on the outer sheath tube 38. At this time, by the action of the compressive force, the outer sheath tube 38 is elastically deformed in a manner to bend to the outside or to the inside.

In addition, in the present embodiment, when the bending section 5 is bend-operated, the front and rear node rings 31 rotate about the rivets 35. At this time, before the two front and rear node rings 31 of the bending tube 30 are rotated to the maximum rotational position (maximum pivotal angle α0) at the non-restriction time, one of the second stepped portions 42 at the rear end portion of the front-side node ring 31 comes in contact with one of the first stepped portions 41 at the front end portion of the rear-side node ring 31, as shown in FIG. 10. By the restriction due to the contact between the one of the second stepped portions 42 of the front-side node ring 31 and the one of the first stepped portions 41 of the rear-side node ring 31, the pivotal angle α between the front and rear node rings 31 is restricted to the predetermined restriction angle α1. This restriction angle α1 is set at the angle that prevents, when the bending section 5 is bend-operated, the outer sheath tube 38 from being bitten between the node rings 31 at the bite limit thickness at which no damage is caused to the outer sheath tube 38. In this case, by the restriction due to the contact between one of the second stepped portions 42 of the front-side node ring 31 and one of the first stepped portions 41 of the rear-side node ring 31, the minimum node ring interval t between the front and rear node rings 31 is set to be greater than the bite limit thickness t0 of the outer sheath tube 38 that is fitted between the node rings 31. Thereby, when the bending section 5 is bend-operated, the outer sheath tube 38 is prevented from being bitten between the node rings 31 and being damaged.

With the above-described structure, the following advantageous effects are obtained. Specifically, in the present embodiment, the first stepped portions 41 for angle restriction for restricting the pivotal angle are formed on both sides of each of the two front-side projection portions 33 on the front end side of the node ring 31. The second stepped portions 42 for angle restriction for restricting the pivotal angle are formed on both sides of each of the two rear-side projection portions 34 on the rear end side of the node ring 31. When the bending section 5 is bend-operated, before the two front and rear node rings 31 of the bending tube 30 are rotated to the maximum rotational position (maximum pivotal angle (t 0) at the non-restriction time, one of the second stepped portions 42 of the front-side node ring 31 comes in contact with one of the first stepped portions 41 of the rear-side node ring 31, as shown in FIG. 10. Thereby, the pivotal angle α1 between the front and rear node rings 31 is restricted to the predetermined restriction angle α1 that prevents, when the bending section 5 is bend-operated, the outer sheath tube 38 from being bitten between the node rings 31 and being damaged. Thus, when the bending section 5 is bend-operated, the outer sheath tube 38 can be prevented from being bitten between the node rings 31 and being damaged.

Therefore, in the present embodiment, when the bending section 5 is bend-operated, the outer sheath tube 38 can be prevented from being bitten between the node rings 31 and being damaged, without providing, unlike the prior art, the protection net between the node rings 31 and the outer sheath tube 38. Thus, compared to the conventional case in which the protection net is provided between the node rings 31 and the cuter sheath tube 38, the manufacture of the insertion section 2 of the endoscope 1 is easier Moreover, it is possible to stabilize the variance in performance of the bending section, which results from the manufacturing process which is performed in the case of providing the protection net and is difficult to automate.

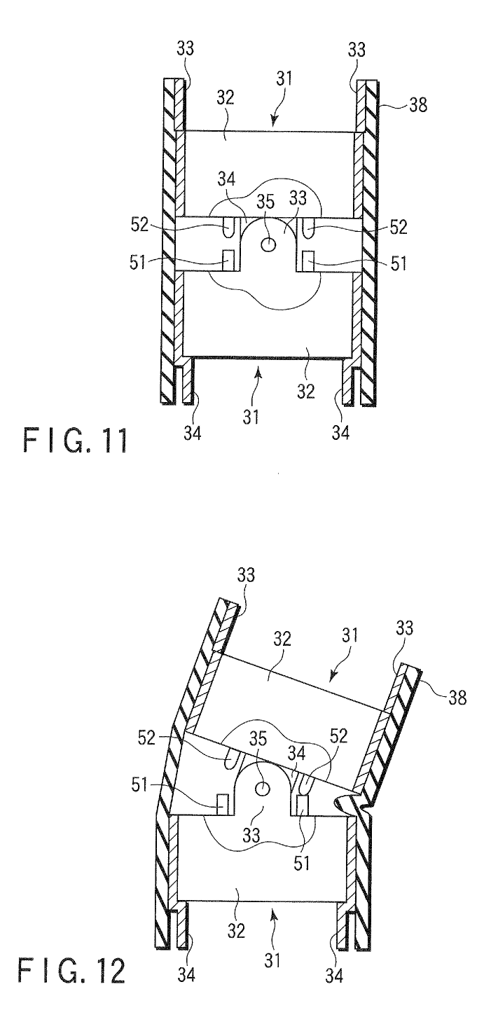

FIG. 11 and FIG. 12 show a second embodiment of the present invention. In the present embodiment, the structure of the bite prevention means of the bending tube 30 of the bending section 5 of the endoscope 1 in the first embodiment (see FIG. 1 to 10) is modified in the following manner. Except for this modified part, this embodiment has the same structure as the bending section 5 of the endoscope 1 of the first embodiment. The parts common to those of the bending section 5 of the endoscope 1 of the first embodiment are denoted by like reference numerals, and a description thereof is omitted.

Specifically, in the present embodiment, first protrusion portions (angle restriction members) 51 for positioning are forwardly projectingly provided on both sides of each of the two front-side projection portions 33 of the node ring 31 on the rear end side of the bending section 5. In addition, second protrusion portions (angle restriction members) 52 for positioning are rearwardly projectingly provided on both sides of each of the two rear-side projection portions 34 of the node ring 31 on the front end side of the bending section 5. The first protrusion portions 51 and second protrusion portions 52 constitute bite prevention means.

FIG. 11 shows the state in which the bending section 5 is kept in the non-bent state. In the state shown in FIG. 11, the two rear-side second protrusion portions 52 of the front-side node ring 31 and the two first protrusion portions 51 of the rear-side node ring 31 are separated in the non-contact state. FIG. 12 shows the state in which the two front-side and rear-side node rings 31 of the bending tube 30 are rotated to the maximum degree when the bending section 5 is bent. In the state shown in FIG. 12, one of the two rear-side second protrusion portions 52 of the front-side node ring 31 and one of the two first protrusion portions 51 of the rear-side node ring 31 are abutted and put in contact. Thereby, the pivot angle α between the front and rear node rings 31, in the case where the two front and rear node rings 31 of the bending tube 30 are rotated to the maximum when the bending section 5 is bent, is restricted to a predetermined restriction angle α1.

Accordingly, in the present embodiment, the bite prevention means is caused to function by abutting, when the bending section 5 is bend-operated, one of the second protrusion portions 52 of the two rear-side projection portions 34 of the front-side node ring 31 of the bending tube 30 and one of the first protrusion portions 51 of the two front-side projection portions 33 of the rear-side node ring 31. The bite prevention means is configured to restrict the pivotal angle α between the front and rear node rings 31 to the predetermined restriction angle α1 in the case where the two front and rear node rings 31 are rotated to the maximum when the bending section 5 is bent.

According to the present embodiment with the above-described structure, the following advantageous effects are obtained. Specifically, in the present embodiment, the first protrusion portions 51 for angle restriction for restricting the pivotal angle are provided on both sides of the two front-side projection portions 33 on the front end side of the node ring 31, and the second protrusion portions 52 for angle restriction for restricting the pivotal angle are provided on both sides of the two rear-side protection portions 34 on the rear end side of the node ring 31. Thereby, when the bending section 5 is bent, before the two front and rear node rings 31 of the bending tube 30 are rotated to the maximum rotational position (maximum pivotal angle α0) at the non-restriction time, one of the second protrusion portions 52 of the front-side node ring 31 comes in contact with one of the first protrusion portions 51 of the rear-side node ring 31, as shown in FIG. 12. Thus, the pivotal angle α between the front and rear node rings 31 is restricted to the predetermined restriction angle α1 that prevents, when the bending section 5 is bend-operated, the outer sheath tube 38 from being bitten between the node rings 31 and being damaged. Hence, when the bending section 5 is bend-operated, the outer sheath tube 38 can be prevented from being bitten between the node rings 31 and being damaged.

Therefore, in the present embodiment, like the first embodiment, when the bending section 5 is bent, the outer sheath tube 38 can be prevented from being bitten between the node rings 31 and being damaged, without providing, unlike the prior art, the protection net between the node rings 31 and the outer sheath tube 38. Thus, compared to the conventional case in which the protection net is provided between the node rings 31 and the outer sheath tube 38, the manufacture of the insertion section 2 of the endoscope 1 is easier. Moreover, it is possible to stabilize the variance in performance of the bending section, which results from the manufacturing process which is performed in the case of providing the protection net and is difficult to automate.

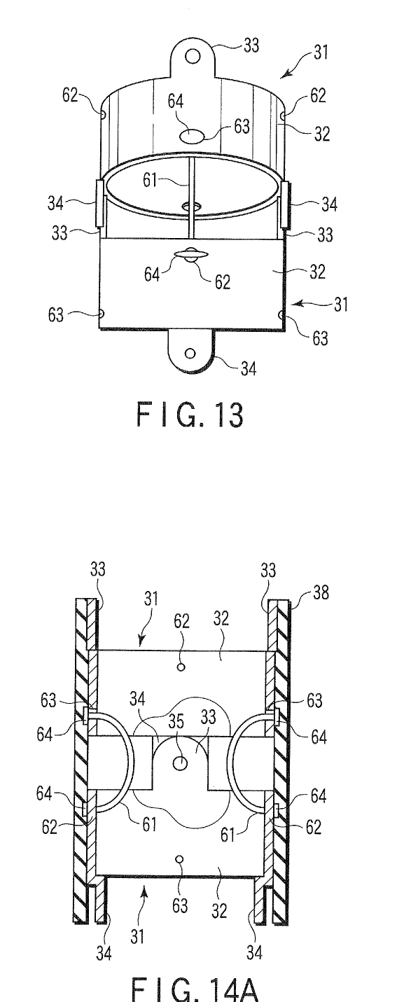

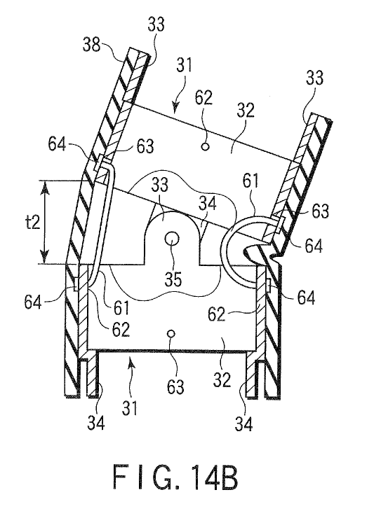

FIG. 13, FIG. 14A and FIG. 14B show a third embodiment of the present invention. In the present embodiment, the structure of the bite prevention means of the bending tube 30 of the bending section 5 of the endoscope 1 in the first embodiment (see FIGS. 1 to 10) is modified in the following manner. Except for this modified part, this embodiment has the same structure as the bending section 5 of the endoscope 1 of the first embodiment. The parts common to those of the bending section 5 of the endoscope 1 of the first embodiment are denoted by like reference numerals, and a description thereof is emitted.

Specifically, the present embodiment has two coupling hands (angle restriction members) 61 which are passed between the front and rear node rings 31. The two coupling bands 61 extend on the opening side of the node rings 31 when the bending section 5 is bend-operated, thereby restricting the maximum node ring interval t2 between the front and rear node rings 31 at the restriction angle α1. The two coupling bands 61 constitute bite prevention means.

Each node ring 31 of the bending tube 30 is formed of, e.g. a resin injection-molding body. First engagement holes 62 are formed at a front end portion of each node ring 31 at intermediate positions between the two front-side projection portions 33 in the circumferential direction of the node ring 31. In addition, second engagement holes 63 are formed at a rear end portion of each node ring 31 at intermediate positions between the two rear-side projection portions 34 in the circumferential direction of the node ring 31.

The coupling band 61 is formed of, e.g. an organic fiber twisted yarn or belt. One end portion of each coupling band 61 is inserted in the second engagement hole 63 at the rear end portion of the front-side node ring 31, and is fixed by a removal prevention member 64 which is fusion-bonded. Similarly, the other end portion of each coupling band 61 is inserted in the first engagement hole 62 at the front end portion of the rear-side node ring 31, and is fixed by a removal prevention member 64 which is fusion-bonded. The other node rings 31 of the bending section 5 are similarly provided with the coupling bands 61 and removal prevention members 64.

FIG. 14A shows the state in which the bending section 5 is kept in the non-bent state. In the state shown in FIG. 14A, the two coupling bands 61, which are passed between the front and rear node rings 31, are held in a loose state. FIG. 14B shows the state in which the two front-side and rear-side node rings 31 of the bending tube 30 are rotated to the maximum degree when the bending section 5 is bent. In the state shown in FIG. 14B, one of the two coupling bands 61 (the coupling band 61 on the opening side of the front and rear node rings 31), which are passed between the front and rear node rings 31, is stretched on the opening side of the front and rear node rings 31. The other coupling band 61 (the coupling band 61 on the closing side of the front and rear node rings 31) is held in a loose state on the closing side of the front and rear node rings 31. In the state in which the coupling band 61 on the opening side of the front and rear node rings 31 is stretched to the stretch limit position, the maximum node ring interval t2 on the opening side of the front and rear node rings 31 is restricted. Thereby, the pivot angle α between the front and rear node rings 31, in the case where the two front and rear node rings 31 of the bending tube 30 are rotated to the maximum when the bending section 5 is bent, is restricted to a predetermined restriction angle α1.

Accordingly, in the present embodiment, the bite prevention means is caused to function by stretching, to the stretch limit position, one of the coupling bands 61, which are passed between the front and rear node rings 31 of the bending section 5, when the bending section 5 is bend-operated. The bite prevention means is configured to restrict the pivotal angle α between the front and rear node rings 31 to the predetermined restriction angle α1 in the case where the two front and rear node rings 31 are rotated to the maximum when the bending section 5 is bent.

According to the present embodiment with the above-described structure, the two coupling bands 61 are provided which are stretched on the opening side of the node rings 31 when the bending section 5 is bend-operated, thereby to restrict the maximum node ring interval t2 between the front and rear node rings 31 at the restriction angle α1. When the bending section 5 is bent, before the two front and rear node rings 31 of the bending tube 30 are rotated to the maximum rotational position (maximum pivotal angle α0) at the non-restriction time, the coupling bands 61 between the front and rear node rings 31 are stretched on the opening side of the node rings 31, as shown in FIG. 14B, thereby to restrict the maximum node ring interval t2 between the front and rear node rings 31. Thereby, the pivotal angle between the front and rear node rings 31 can be restricted to the predetermined restriction angle α1. Hence, when the bending section 5 is bend-operated, the outer sheath tube 38 can be prevented from being bitten between the node rings 31 and being damaged.

Therefore, in the present embodiment, like the first embodiment, when the bending section 5 is bent, the outer sheath tube 38 can be prevented from being bitten between the node rings 31 and being damaged, without providing, unlike the prior art, the protection net between the node rings 31 and the outer sheath tube 38. Thus, compared to the conventional case in which the protection net is provided between the node rings 31 and the outer sheath tube 38, the manufacture of the insertion section 2 of the endoscope 1 is easier. Moreover, it is possible to stabilize the variance in performance of the bending section, which results from the manufacturing process which is performed in the case of providing the protection net and is difficult to automate.

Needless to say, the present invention is not limited to the above-described embodiments, and various modifications may be made without departing from the spirit of the invention.

Next, other characteristic technical matters of the present invention are noted below.

### Note

(Item 1) An endoscope bending section in which an elastic circular-cylindrical outer sheath is directly fitted over an outer periphery of a bending tube in which node rings are coupled, the node ring having a shape or a member for restricting a pivotal angle between the node rings in such a manner that a minimum node ring Interval is set to be a bite limit thickness or more, at which the outer sheath that is fitted between the node rings is not damaged.

(Item 2) The endoscope bending section according to item 1, wherein the node rings have stepped (protrusion) portions which are put in contact at a restriction pivotal angle in both directions at the node rings (hinge bases of the node rings) which are mutually coupled and pivotally moved.

(Item 3) The endoscope bending section according to item 1, wherein the node rings have coupling bands (chains) which are passed between the node rings that are mutually coupled and pivotally moved, and which are stretched on an opening side at a restriction pivotal angle in both directions.

The present invention is effective in a technical field of manufacturing a bending section of an endoscope in which a bendable bending section is provided at a distal end portion of an insertion section which is inserted into the body.

What is claimed is:

1. A bending section structure of an endoscope, comprising a bending section including a bending tube in which a plurality of node rings are juxtaposed along an insertion direction of an endoscope insertion section and are coupled such that front and rear said node rings are rotatable about support shaft portions, and an outer sheath which is formed of an elastic material in a circular cylindrical shape and is directly fitted over an outer periphery of the bending tube,
wherein the bending section structure of the endoscope includes a bending wire for pulling and operating the bending section, and the node rings of the bending tube are rotated and operated about the support shaft portions by the pulling operation of the bending wire, thereby bending the bending section,
the node ring has bite prevention means for restricting, to a predetermined restriction angle, a pivotal angle between the front and rear node rings which rotate about the support shaft portions when the bend section is bend-operated, and
the restriction angle is so set as not to exceed a bite limit thickness at which the outer sheath is not damaged when the outer sheath is bitten between the node rings.

2. The bending section structure of the endoscope according to claim 1, wherein the rode ring includes an annular node ring body, a front-side hinge base which is forwardly projectingly provided at a front end portion of the node ring body, and a rear-side hinge base which is rearwardly projectingly provided at a rear end portion of the node ring body,
the support shaft portion includes a pivotal support shaft which is pivotally coupled to an overlap part between the rear-side hinge base of the node ring, which is disposed on a front side, and the front-side hinge base of the node ring, which is disposed on a rear side,
the bite prevention means includes an angle restriction member which is provided on the node ring, and
the angle restriction member restricts, to the restriction angle, the pivotal angle between the front and rear node rings which rotate about the pivotal support shafts when the bend section is bend-operated.

3. The bending section structure of the endoscope according to claim 2, wherein the angle restriction member restricts the restriction angle such that a minimum node ring interval between the front and rear node rings becomes greater than a bite thickness of the outer sheath that is fitted between the node rings when the bending section is bend-operated.

4. The bending section structure of the endoscope according to claim 2, wherein the angle restriction member includes, at least on one of the rear-side hinge base and the front-side hinge base, an abutment portion at which the rear-side hinge base and the front-side hinge base are abutted at the restriction angle when the bending section is bend-operated.

5. The bending section structure of the endoscope according to claim 4, wherein the abutment portion includes, at each of the rear-side hinge base and the front-side hinge base, a stepped portion for abutment at the restriction angle at a time when the bending section is bend-operated.

6. The bending section structure of the endoscope according to claim 2, wherein the angle restriction member includes a coupling band which is passed between the front and rear node rings, and
the coupling band is stretched on an opening side of the node rings when the bending section is bend-operated, thereby restricting a maximum node ring interval between the front and rear node rings at the restriction angle.

7. The bending section structure of the endoscope according to claim 2, wherein first protrusion portions for positioning are forwardly projectingly provided on both sides of the front-side hinge base of the node ring, and second protrusion portions for positioning are rearwardly projectingly provided on both sides of the rear-side hinge base of the node ring, and
the angle restriction member restricts the restriction angle by putting the second protrusion portion of the rear-side hinge base and the first protrusion portion of the front-side hinge base at the restriction angle when the bending section is bend-operated.
